(19) United States
(12) Patent Application Publication
(10) Pub. No.: US 2021/0282842 A1
(43) Pub. Date: Sep. 16, 2021

(54) SURGICAL INSTRUMENT INCLUDING A TRIGGER LOCKOUT MECHANISM AND SURGICAL METHODS

(71) Applicant: Covidien LP, Mansfield, MA (US)

(72) Inventors: Rajanikanth Mandula, Hyderabad (IN); Ravi Sekhar Gutti, Hyderabad (IN)

(21) Appl. No.: 16/816,985

(22) Filed: Mar. 12, 2020

(51) Int. Cl. A61B 18/14

(52) U.S. Cl. CPC A61B 18/1445; A61B 2018/00922

(57) ABSTRACT

An electrosurgical instrument includes a housing, a movable handle movable relative to the housing along an actuation path from an initial position to a grasping position to an activated position, an in-line activation switch disposed on the housing along the actuation path of the movable handle such that movement of the movable handle from the grasping position to the activated position activates the in-line activation switch, a trigger coupled the knife and movable relative to the housing from an un-actuated position to an actuated position, and a knife lockout operably associated with the movable handle and the trigger. The knife lockout is configured to inhibit actuation of the trigger from the un-actuated position to the actuated position until the movable handle reaches the activated position.

### FIELD

The present disclosure relates to surgical instruments and, more particularly, to surgical instruments including trigger lockout mechanisms and surgical methods.

### BACKGROUND

A surgical forceps is a pliers-like surgical instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is treated, the surgeon has to accurately sever the treated tissue. Accordingly, many electrosurgical forceps are designed to incorporate a knife or cutting member utilized to effectively sever the treated tissue.

### SUMMARY

As used herein, the term “distal” refers to the portion that is being described which is further from a user, while the term “proximal” refers to the portion that is being described which is closer to a user. Further, to the extent consistent, any or all of the aspects detailed herein may be used in conjunction with any or all of the other aspects detailed herein.

Provided in accordance with aspects of the present disclosure is an electrosurgical instrument including a housing, a movable handle movable relative to the housing along an actuation path from an initial position to a grasping position to an activated position, an in-line activation switch disposed on the housing along the actuation path of the movable handle such that movement of the movable handle from the grasping position to the activated position activates the in-line activation switch, a trigger coupled the knife and movable relative to the housing from an un-actuated position to an actuated position, and a knife lockout operably associated with the movable handle and the trigger and configured to inhibit actuation of the trigger from the un-actuated position to the actuated position until the movable handle reaches the activated position.

In an aspect of the present disclosure, the electrosurgical instrument further includes a shaft extending distally from the housing and an end effector assembly extending distally from the shaft. The end effector assembly includes first and second jaw members at least one of which is movable relative to the other from a spaced-apart position to an approximated position to grasp tissue therebetween. Movement of the movable handle from the initial position to the grasping position moves the at least one of the first or second jaw members from the spaced-apart position to the approximated position.

In another aspect of the present disclosure, the electrosurgical instrument further includes a knife selectively deployable from a retracted position to an extended position wherein the knife extends at least partially between the first and second jaw members to cut tissue grasped therebetween. The trigger is coupled the knife and movable relative to the housing from an un-actuated position to an actuated position to deploy the knife from the retracted position to the extended position.

In yet another aspect of the present disclosure, the in-line activation switch is electrically coupled between electrically-conductive surfaces of the first and second jaw members and a source of electrosurgical energy such that activation of the in-line activation switch initiates a supply of electrosurgical energy from the source of electrosurgical energy to the electrically-conductive surfaces.

In still another aspect of the present disclosure, the knife lockout includes a first component engaged with the movable handle and second component engaged with the trigger.

In another aspect of the present disclosure, the knife lockout includes a slot and a post. The knife lockout is disposed in a locked condition inhibiting actuation of the trigger from the un-actuated position to the actuated position when the post is disposed within the slot and an unlocked condition permitting actuation of the trigger from the un-actuated position to the actuated position when the post is withdrawn from the slot.

In still another aspect of the present disclosure, the slot is defined within an arm extending from one of the movable handle or the trigger and the post extends from the other of the movable handle or the trigger.

In yet another aspect of the present disclosure, the knife lockout includes an arm extending from the movable handle towards the trigger. In such aspects, the arm may define a slot having an open end and a closed end or, in other aspects, two closed ends. Further, the trigger may include a post wherein: in the initial position of the movable handle, the post is received within the slot to inhibit actuation of the trigger from the un-actuated position to the actuated position; in the grasping position of the movable handle, the post is received within the slot to inhibit actuation of the trigger from the un-actuated position to the actuated position; and in the activated position, the post is withdrawn from the slot to permit actuation of the trigger from the un-actuated position to the actuated position.

A method of operating an electrosurgical instrument provided in accordance with aspects of the present disclosure includes actuating a movable handle of electrosurgical instrument from an initial position to a grasping position to grasp tissue between first and second jaw members of the electrosurgical instrument and actuating the movable handle from the grasping position to an activated position to initiate a supply energy to the first and second jaw members to treat the tissue grasped therebetween. Actuation the movable handle to the activated position unlocks a trigger of the electrosurgical instrument. The method further includes actuating the (now unlocked) trigger from an un-actuated position to an actuated position to deploy a knife between the first and second jaw members to cut the grasped and treated tissue.

In an aspect of the present disclosure, the trigger is locked in the un-actuated position prior to the movable handle reaching the activated position.

In another aspect of the present disclosure, actuating the movable handle from the grasping position to the activated position moves the movable handle into contact with an in-line activation switch to activate the in-line activation switch thereby initiating the supply of energy to the first and second jaw members.

In still another aspect of the present disclosure, the supply of energy to the first and second jaw members is automatically terminated once it is determined that the grasped tissue is sufficiently treated.

In yet another aspect of the present disclosure, the trigger is actuated from the un-actuated position to the actuated position subsequent to termination of the supply of energy.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of an electrosurgical forceps provided in accordance with the present disclosure;

FIG. 2A is an enlarged, perspective view of an end effector assembly of the electrosurgical forceps of FIG. 1 wherein first and second jaw members of the end effector assembly are disposed in a spaced-apart position;

FIG. 2B is an enlarged, perspective view of the end effector assembly of FIG. 2A wherein the first and second jaw members are disposed in an approximated position;

FIG. 3A is a side view of a proximal portion of the electrosurgical forceps of FIG. 1 with a portion of a housing removed to illustrate internal components, wherein a movable handle is disposed in an initial position and a trigger is disposed in an un-actuated position;

FIG. 3B is a side view of the proximal portion of the electrosurgical forceps shown in FIG. 3A with the portion of the housing removed to illustrate internal components, wherein the movable handle is disposed in a grasping position and the trigger is disposed in the un-actuated position;

FIG. 3C is a side view of the proximal portion of the electrosurgical forceps shown in FIG. 3A with the portion of the housing removed to illustrate internal components, wherein the movable handle is disposed in an activated position and the trigger is disposed in the un-actuated position; and

FIG. 3D is a side view of the proximal portion of the electrosurgical forceps shown in FIG. 3A with the portion of the housing removed to illustrate internal components, wherein the movable handle is disposed in the activated position and the trigger is disposed in an actuated position.

### DETAILED DESCRIPTION

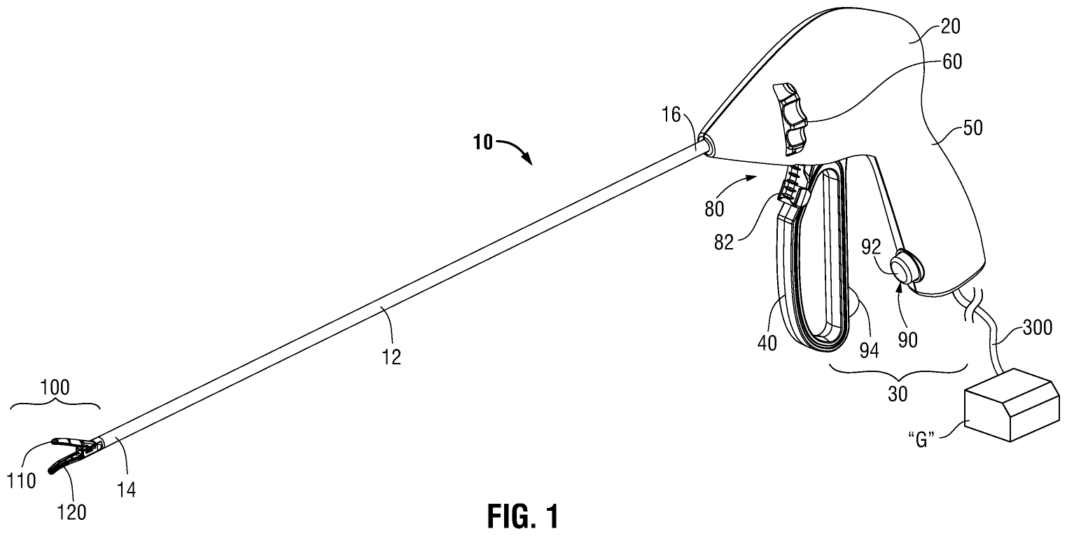

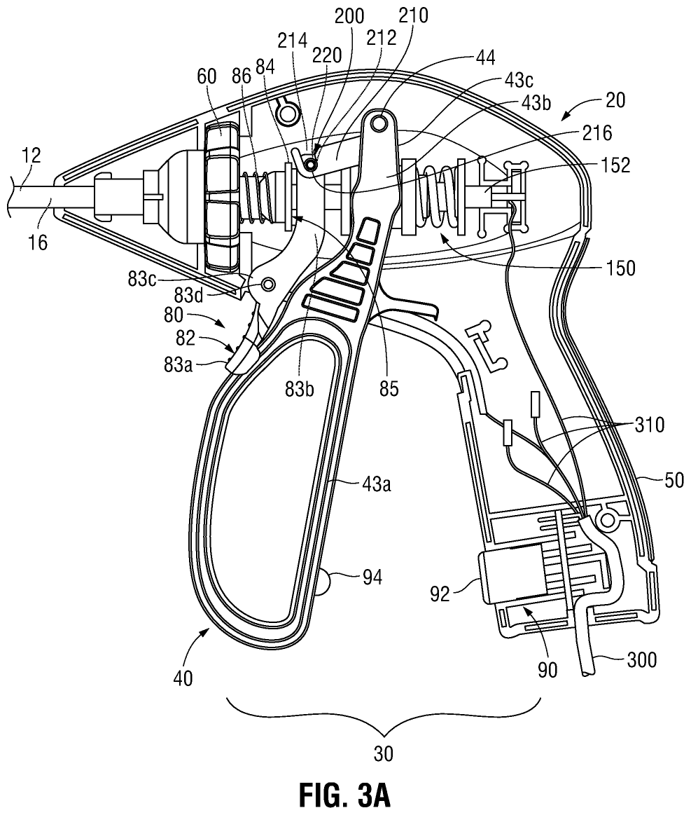

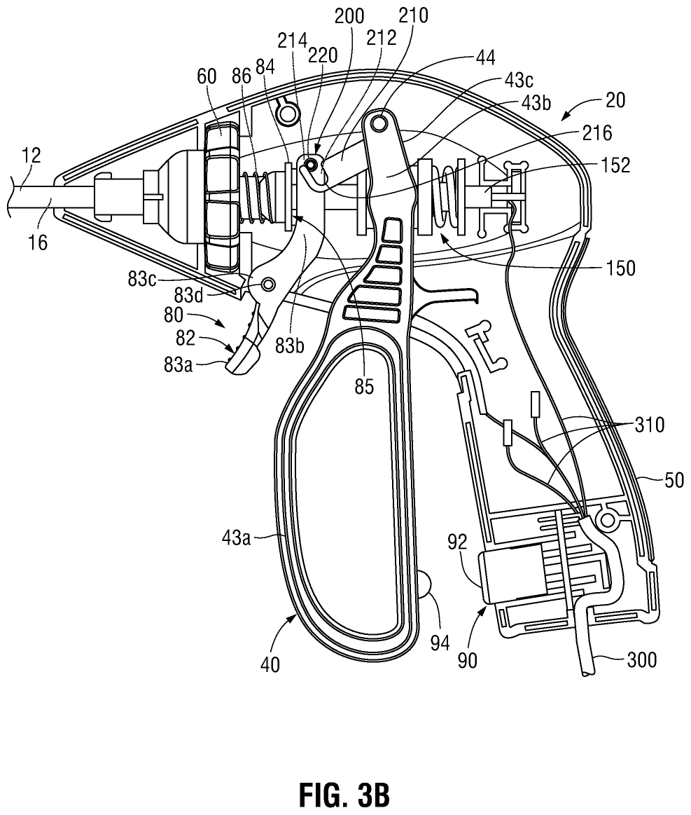

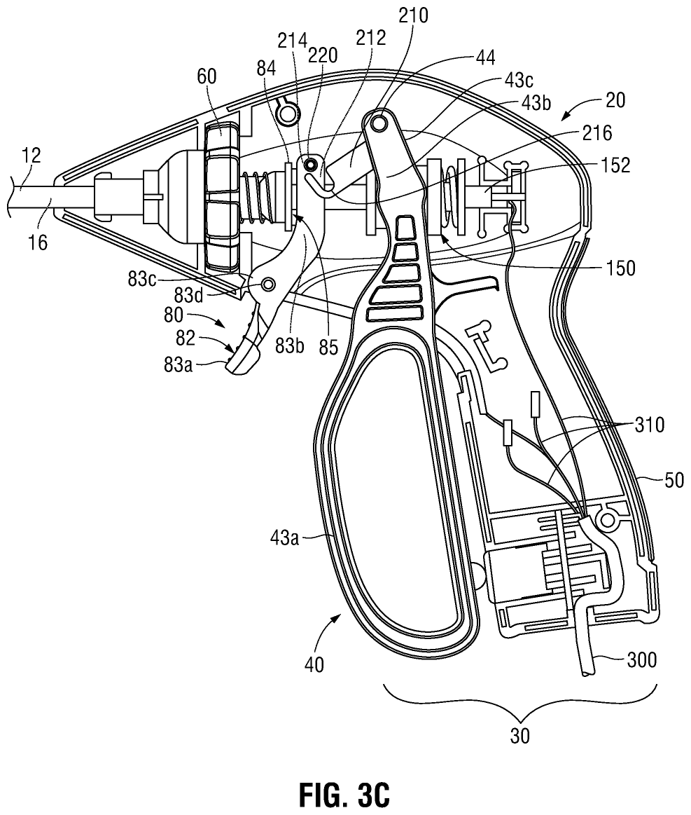

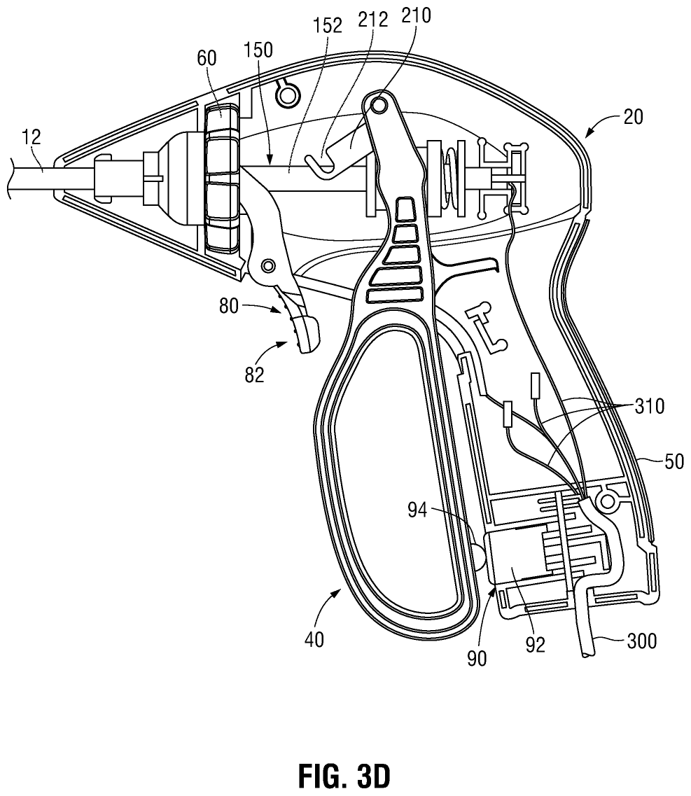

Referring to FIG. 1, a surgical instrument provided in accordance with the present disclosure is shown configured as a bipolar electrosurgical forceps 10 for use in connection with endoscopic surgical procedures, although the present disclosure is equally applicable for use with other surgical instruments such as those for use in endoscopic and/or traditional open surgical procedures. Forceps 10 generally includes a housing 20, a handle assembly 30, a rotating assembly 60, a trigger assembly 80, an activation assembly 90, and an end effector assembly 100 including first and second jaw members 110, 120. Forceps 10 additionally includes a trigger lockout mechanism 200 (see FIGS. 3A-3D) associated with handle assembly 30 and trigger assembly 80 to inhibit actuation of trigger 82 of trigger assembly 80 until movable handle 40 of handle assembly 30 is moved to an activated position to initiate a supply of energy to end effector assembly 100, as detailed below.

Forceps 10 further includes a shaft 12 having a distal end portion 14 configured to engage (directly or indirectly) end effector assembly 100 and a proximal end portion 16 that engages (directly or indirectly) housing 20. Rotating assembly 60 is rotatable in either direction to rotate shaft 12 and end effector assembly 100 relative to housing 20 in either direction. Housing 20 houses the internal working components of forceps 10.

An electrosurgical cable 300 connects forceps 10 to an electrosurgical generator “G” or other suitable energy source, although forceps 10 may alternatively be configured as a handheld instrument incorporating energy-generating and/or power components thereon or therein. Cable 300 includes wires 310 (FIGS. 3A-3D) extending therethrough, into housing 20, and through shaft 12, to ultimately connect electrosurgical generator “G” to jaw member 110 and/or jaw member 120 of end effector assembly 100. Activation button 92 of activation assembly 90 is disposed on housing 20 are electrically coupled between end effector assembly 100 and cable 300 to enable the selective supply of energy to jaw member 110 and/or jaw member 120, e.g., upon activation of activation button 92. However, other suitable electrical connections and/or configurations for supplying electrosurgical energy to jaw member 110 and/or jaw member 120 may alternatively be provided, as may other suitable forms of energy, e.g., ultrasonic energy, microwave energy, light energy, thermal energy, etc.

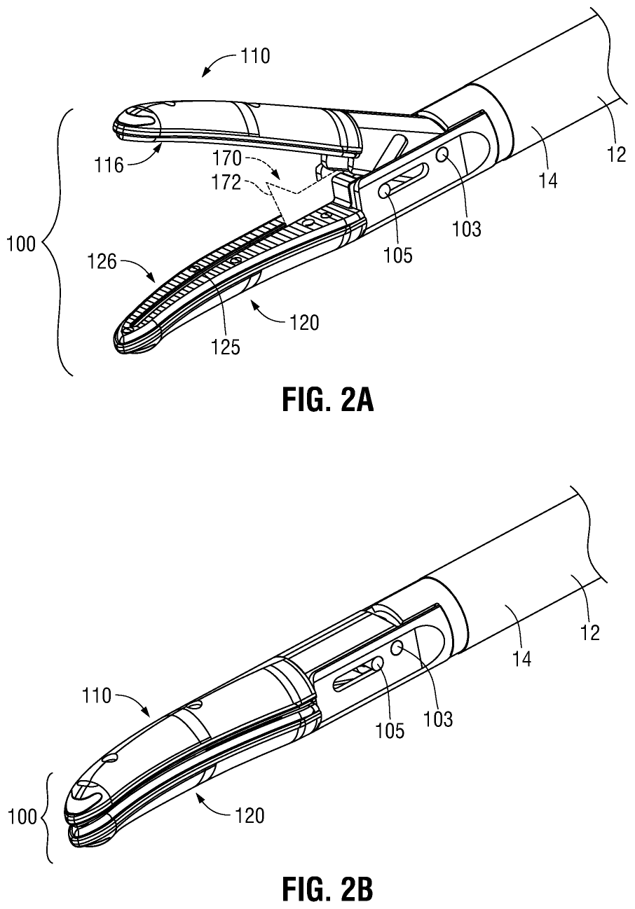

Referring momentarily to FIGS. 2A and 3A, forceps 10 additionally includes a knife assembly 170 operably coupled to trigger assembly 80 and extending through housing 20 and shaft 12. One or both of jaw members 110, 120 defines a knife channel 125 (FIG. 2A) configured to permit reciprocation of a knife blade 172 of knife assembly 170 therethrough, e.g., in response to actuation of trigger 82 of trigger assembly 80. Knife assembly 170 further includes a knife bar (not shown) extending through shaft 12 and operably coupling knife blade 172 with a proximal mandrel 84 of trigger assembly 80 with such that translation of proximal mandrel 84 through housing 20 translates the knife bar through shaft 12 to thereby translate knife blade 172 through knife channel 125, e.g., between a retracted position and an extended position relative to jaw members 110, 120. Trigger 82 of trigger assembly 80 is operably coupled to proximal mandrel 84 of trigger assembly 80 such that actuation of trigger 82 translates proximal mandrel 84. Trigger assembly 80 is described in greater detail below, as is trigger lockout mechanism 200 (see FIGS. 3A-3D).

With reference to FIGS. 2A and 2B, in conjunction with FIG. 1, end effector assembly 100, as noted above, is disposed at distal end portion 14 of shaft 12 and includes a pair of jaw members 110 and 120 pivotable between a spaced-apart position and an approximated position for grasping tissue therebetween. End effector assembly 100 is designed as a unilateral assembly, e.g., wherein one of the jaw members 120 is fixed relative to shaft 12 and the other jaw member 110 is movable relative to both shaft 12 and the fixed jaw member 120. However, end effector assembly 100 may alternatively be configured as a bilateral assembly, e.g., wherein both jaw member 110 and jaw member 120 are movable relative to one another and with respect to shaft 12.

Each jaw member 110, 120 of end effector assembly 100 includes an electrically-conductive tissue-contacting surface 116, 126. Tissue-contacting surfaces 116 are positioned to oppose one another for grasping and treating tissue. More specifically, tissue-contacting surfaces 116, 126 are electrically coupled to the generator “G,” e.g., via cable 300, and activation button 92 to enable the selective supply of energy thereto for conduction through tissue grasped therebetween, e.g., upon activation of activation button 92. One or both of tissue-contacting surfaces 116, 126 may include one or more stop members (not shown) extending therefrom to define a minimum gap distance between electrically-conductive tissue-contacting surfaces 116, 126 in the approximated position of jaw members 110, 120, facilitate grasping of tissue, and/or inhibit shorting between electrically-conductive tissue-contacting surfaces 116, 126. The stop member(s) may be formed at least partially from an electrically-insulative material or may be effectively insulative by electrically isolating the stop member(s) from one or both of the electrically-conductive tissue-contacting surfaces 116, 126.

A pivot pin 103 of end effector assembly 100 extends transversely through aligned apertures defined within jaw members 110, 120 and shaft 12 to pivotably couple jaw member 110 to jaw member 120 and shaft 12. A cam pin 105 of end effector assembly 100 extends transversely through cam slots defined within jaw members 110, 120 and is operably engaged with a distal end portion of a drive bar 152 (FIG. 3A) of a drive assembly 150 (FIG. 3A) such that longitudinal translation of drive bar 152 (FIGS. 4A and 4B) through shaft 12 translates cam pin 105 relative to jaw members 110, 120. More specifically, proximal translation of cam pin 105 relative to jaw members 110, 120 urges cam pin 105 proximally through the cam slots to thereby pivot jaw members 110, 120 from the spaced-apart position towards the approximated position, although drive assembly 150 (FIG. 3A) and end effector assembly 100 may alternatively be configured such that distal translation of cam pin 105 pivots jaw members 110, 120 from the spaced-apart position towards the approximated position. One suitable drive assembly is described in greater detail, for example, in U.S. Pat. No. 9,655,673, the entire contents of which are hereby incorporated herein by reference.

Referring to FIGS. 1-3C, handle assembly 30 includes a fixed handle 50 and a movable handle 40. Fixed handle 50 is integrally associated with housing 20 and movable handle 40 is movable relative to fixed handle 50. Movable handle 40 is ultimately connected to drive assembly 150 that, together, mechanically cooperate to impart movement of jaw members 110 and 120 between the spaced-apart and approximated positions to grasp tissue between electrically-conductive surfaces 116, 126, respectively. More specifically, movable handle 40 includes a manipulation portion 43a extending from housing 20 to enable manual manipulation thereof by a user. Movable handle 40 further includes a drive portion 43b and a pivot portion 43c. Movable handle 40 is monolithically formed from a single piece of material or is otherwise formed, e.g., via fixed engagements, such that manipulation portion 43a, drive portion 43b, and pivot portion 43c are fixed relative to one another. Pivot portion 43c is pivotably coupled within housing 20, e.g., via a pivot pin 44. Pivot portion 43c is disposed at one end of movable handle 40 with drive portion 43b extending from pivot portion 43c and manipulation portion 43a extending from drive portion 43b such that movement of manipulation portion 43a in one direction, e.g., proximally, urges drive portion 43b in the same direction, e.g., proximally. However, other configurations are also contemplated.

As a result of the above-detailed configuration, pivoting of movable handle 40 relative to fixed handle 50 from an initial position (FIG. 3A) towards a grasping position (FIG. 3B) pivots jaw members 110, 120 from the spaced-apart position (FIG. 2A) towards the approximated position (FIG. 2B) to grasp tissue therebetween. On the other hand, when movable handle 40 is released or returned towards the initial position relative to fixed handle 50, jaw members 110, 120 are returned towards the spaced-apart position. In embodiments, drive assembly 150 may include a force-limiting spring or other suitable mechanism configured to limit the jaw force applied by jaw members 110, 120 to tissue grasped therebetween to within a pre-determined range. In such embodiments, handle assembly 30 and drive assembly 150 may be configured such that the pre-determined force range is reached upon movement of movable handle 40 to the grasping position. Thereafter, the force-limiting spring decouples movable handle 40 from drive assembly 150 such that further actuation of movable handle 40, e.g., from the grasping position to the activated position does not alter the position of jaw members 110, 120 and, thus, does not apply additional jaw force to tissue grasped between jaw members 110, 120.

Fixed handle 50 operably supports activation button 92 of activation assembly 90 thereon in an in-line position, wherein activation button 92 is disposed in the actuation path of movable handle 40. In this manner, upon pivoting of movable handle 40 relative to fixed handle 50 from the initial position (FIG. 3A), through the grasping position (FIG. 3B), to an activated position (FIG. 3C), protrusion 94 of movable handle 40 is urged into contact with activation button 92 to thereby activate activation button 92 and initiate the supply of energy to electrically-conductive surfaces 116, 126, e.g., to treat tissue grasped therebetween. Upon activation of activation button 92, energy may be supplied to electrically-conductive surfaces 116, 126 in any suitable manner such as, for example, in accordance with a feedback-based energy delivery algorithm wherein once tissue grasped between electrically-conductive surfaces 116, 126 is determined to be sufficiently treated, e.g., sealed, the supply of energy to electrically-conductive surfaces 116, 126 is automatically terminated (even if activation button 92 remains depressed). However, other suitable energy delivery and/or control configurations are also contemplated.

With reference to FIGS. 1-2B and 3A-3D, as noted above, trigger assembly 80 is operably coupled to knife blade 172 of knife assembly 170 by way of engagement between proximal mandrel 84 of trigger assembly 80 and the knife bar (not shown) of knife assembly 170. More specifically, trigger 82 of trigger assembly 80 is selectively actuatable, e.g., from an un-actuated positon (FIGS. 3A-3C) to an actuated position (FIG. 3D), to deploy knife blade 172 distally through jaw members 110, 120 to cut tissue grasped between electrically-conductive surfaces 116, 126.

Trigger assembly 80 includes trigger 82 and proximal mandrel 84. Trigger 82 includes a manipulation portion 83a extending from housing 20 to enable manual manipulation thereof by a user. Trigger 82 further includes a drive portion 83b and a pivot portion 83c. Trigger 82 is monolithically formed from a single piece of material or is otherwise formed, e.g., via fixed engagements, such that manipulation portion 83a, drive portion 83b, and pivot portion 83c are fixed relative to one another. Pivot portion 83c is pivotably coupled within housing 20 via receipt of a pair of pivot posts 83d extending outwardly from opposite sides of pivot portion 83c within corresponding apertures (not shown) defined on opposed interior sides of housing 20. Pivot portion 83c is disposed between manipulation portion 83a, which extends from housing 20, and drive portion 83b, which is disposed within housing 20, such that movement of manipulation portion 83a in one direction, e.g., proximally, urges drive portion 83b in the opposite direction, e.g., distally.

Proximal mandrel 84 is slidably disposed about drive bar 152 and, as noted above, is engaged with the knife bar (not shown) of knife assembly 170. More specifically, a pin (not shown) extending transversely within proximal mandrel 84 and engaged therewith on either side of the pin may extend through a slot (not shown) defined within drive bar 152 and engage the knife bar within drive bar 152. In this manner, translation of proximal mandrel 84 about drive bar 152 translates the pin through the slot to thereby translate the knife bar through drive bar 152 and relative to shaft 12 to, as noted above, deploy and retract knife 172 relative to end effector assembly 100.

Drive portion 83b of trigger 82 extends upwardly from pivot portion 83c further into housing 20, extending beyond drive bar 152 on at least one side of drive bar 152. In embodiments, drive portion 83b of trigger 82 is bifurcated to extend beyond drive bar 152 on both sides thereof. Drive portion 83b is positioned adjacent, on a proximal side thereof, a proximally-facing surface 85 of proximal mandrel 84. As such, pivoting manipulation portion 83a proximally urges drive portion 83b distally into the proximally-facing surface 85 of proximal mandrel 84, thereby urging proximal mandrel 84 distally about drive bar 152 to deploy knife 172 relative to end effector assembly 100.

A biasing spring 86 of trigger assembly 80 is disposed about drive bar 152 and positioned between a stop (not shown) longitudinally fixed relative to housing 20 and proximal mandrel 84 to bias proximal mandrel 84 towards a more-proximal positon. Thus, in the absence of sufficient distal urging, e.g., from drive portion 83b of trigger 82, to translate or retain proximal mandrel 84 to a more-distal position (corresponding to the deployed position of knife 172), proximal mandrel 84 is maintained or returned in the more-proximal positon (corresponding to the retracted positon of knife 172).

Continuing with reference to FIGS. 1-2B and 3A-3D, trigger lockout mechanism 200 (see FIGS. 3A-3D), as noted above, is associated with handle assembly 30 and trigger assembly 80 to inhibit actuation of trigger 82 of trigger assembly 80 until movable handle 40 of handle assembly 30 is moved to the activated position (FIG. 3C) whereby, as noted above, protrusion 94 of movable handle 40 is urged into contact with activation button 92 to thereby activate activation button 92 and initiate the supply of energy to electrically-conductive surfaces 116, 126, e.g., to treat tissue grasped therebetween. Trigger lockout mechanism 200 thus inhibits premature actuation of knife 172 such as, for example, where knife 172 is deployed to cut tissue that has yet to be sufficiently treated.

Trigger lockout mechanism 200 includes an arm 210 extending distally from movable handle 40 on one or both sides thereof and a post 220 extending transversely from one side, both sides, or between trigger 82. More specifically, arm(s) 210 extends distally from pivot portion 43c of movable handle 40 at a position spaced-apart from pivot pin 44, e.g., between pivot pin 44 and drive portion 43b of movable handle 40. One arm 210 may be provided on one side of drive bar 152 or two arms 210 may be provided one on each side of drive bar 152. Post(s) 220 is disposed above drive bar 152 (although, in other embodiments, such could be below the drive bar) and extends transversely from drive portion 83b of trigger 82. One post 210 may be provided on one side of drive bar 152, e.g., where drive portion 83b only extends on one side of drive bar 152, two posts 220 may be provided, e.g., one extending from each of the bifurcated segments of drive portion 83b in bifurcated configurations, or one post 220 may extend between bifurcated segments of drive portion 83b in bifurcated configurations. Although multiple arms 210 and/or posts 220 are contemplated, as noted above, trigger lockout mechanism 200 is detailed below with reference to one arm 210 and one post 220 for the simplicity of reference in the singular.

Arm 210 defines a slot 212 having an open distal end 214 and a closed proximal end 216. Open distal end 214 of slot 212 enables entry of post 220 into slot 212 and exit of post 220 from slot 212. Slot 212 may define an arcuate configuration having an arc of curvature generally complementary to the arc of curvature defined by pivoting of movable handle 40 between the initial, grasping, and actuated positions (FIGS. 3A, 3B, and 3C, respectively). Accordingly, with post 220 received within slot 212, post 220 is moved along slot 212 upon pivoting of movable handle 40 relative to fixed handle 50 without interfering with the pivoting of movable handle 40. Other configurations that enable post 220 to move along slot 212 upon pivoting of movable handle 40 relative to fixed handle 50 without interfering with the pivoting of movable handle 40 are also contemplated. The curvature, orientation, and/or configuration of slot 212 also inhibits post 220 from moving distally from slot 212 out of open distal end 214 thereof upon pivoting of trigger 82. That is, the inner surface of arm 210 that defines slot 212 blocks distal movement of post 220 when post 220 is received within slot 212 such that pivoting of trigger 82 from the un-actuated position is inhibited when post 220 is received within slot 212. Thus, deployment of knife 172 (FIG. 2A) is inhibited when post 220 is received within slot 212.

As illustrated in FIG. 3A, in the initial position of movable handle 40, trigger 82 is disposed in the un-actuated position and post 220 is disposed within slot 212 at or adjacent closed proximal end 216 thereof. Thus, trigger 82 is locked in the un-actuated position as any attempted actuation of trigger 82 would drive post 220 into the inner surface of arm 210 that defines slot 212 to inhibit such actuation.

Referring to FIG. 3B, as movable handle 40 is moved from the initial position to the grasping position, while trigger 82 remains in the un-actuated position, arm 210 is moved relative to post 220 such that post 220 moves distally through slot 212 towards open distal end 214 thereof. However, in the grasping position of movable handle 40, post 220 does not reach the open distal end 214 of slot 212 but, rather, remains disposed within slot 212. Thus, trigger 82 is still locked in the un-actuated position as any attempted actuation of trigger 82 would drive post 220 into the inner surface of arm 210 that defines slot 212 to inhibit such actuation.

With reference to FIG. 3C, when it is desired to treat tissue grasped between jaw members 110, 120 (FIGS. 2A-2B), movable handle 40 is moved from the grasping position to the activated position, wherein protrusion 94 of movable handle 40 is urged into contact with activation button 92 to thereby activate activation button 92 and initiate the supply of energy to electrically-conductive surfaces 116, 126 of jaw members 110, 120 (see FIGS. 2A-2B), e.g., to treat tissue grasped therebetween. With movable handle 40 disposed in the activated position, arm 210 is positioned such that post 220 is withdrawn from open distal end 214 of slot 212. Thus, referring also to FIG. 3D, post is 220 is free from interference, thereby enabling trigger 82 to be pivoted from the un-actuated position to the actuated position to deploy knife blade 172 distally through jaw members 110, 120, e.g., to cut the treated tissue grasped between electrically-conductive surfaces 116, 126 of jaw members 110, 120 (see FIGS. 2A and 2B).

Upon release or return of trigger 82 and movable handle 40, trigger 82 is returned to the un-actuated position and movable handle 40 is returned to the initial position wherein, referring back to FIG. 3A, post 220 is returned to slot 212 and positioned at or adjacent closed proximal end 216 thereof to once again lockout actuation of trigger 82. A return spring (not shown) may be provided to facilitate return of trigger 82; in embodiments, arm 210 may additionally or alternatively facilitate return of trigger 82.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An electrosurgical instrument, comprising:
a housing;
a movable handle movable relative to the housing along an actuation path from an initial position to a grasping position to an activated position;
an in-line activation switch disposed on the housing along the actuation path of the movable handle such that movement of the movable handle from the grasping position to the activated position activates the in-line activation switch;
a trigger coupled the knife and movable relative to the housing from an un-actuated position to an actuated position; and
a knife lockout operably associated with the movable handle and the trigger and configured to inhibit actuation of the trigger from the un-actuated position to the actuated position until the movable handle reaches the activated position.

2. The electrosurgical instrument according to claim 1, further comprising:
a shaft extending distally from the housing; and
an end effector assembly extending distally from the shaft and including first and second jaw members, at least one of the first or second jaw members movable relative to the other from a spaced-apart position to an approximated position to grasp tissue therebetween,
wherein movement of the movable handle from the initial position to the grasping position moves the at least one of the first or second jaw members from the spaced-apart position to the approximated position.

3. The electrosurgical instrument according to claim 2, further comprising:
a knife selectively deployable from a retracted position to an extended position wherein the knife extends at least partially between the first and second jaw members to cut tissue grasped therebetween,
wherein the trigger is coupled the knife and movable relative to the housing from an un-actuated position to an actuated position to deploy the knife from the retracted position to the extended position.

4. The electrosurgical instrument according to claim 2, wherein the in-line activation switch is electrically coupled between electrically-conductive surfaces of the first and second jaw members and a source of electrosurgical energy such that activation of the in-line activation switch initiates a supply of electrosurgical energy from the source of electrosurgical energy to the electrically-conductive surfaces.

5. The electrosurgical instrument according to claim 1, wherein the knife lockout includes a first component engaged with the movable handle and second component engaged with the trigger.

6. The electrosurgical instrument according to claim 1, wherein the knife lockout includes a slot and a post, wherein the knife lockout is disposed in a locked condition inhibiting actuation of the trigger from the un-actuated position to the actuated position when the post is disposed within the slot and an unlocked condition permitting actuation of the trigger from the un-actuated position to the actuated position when the post is withdrawn from the slot.

7. The electrosurgical instrument according to claim 6, wherein the slot is defined within an arm extending from one of the movable handle or the trigger and wherein the post extends from the other of the movable handle or the trigger.

8. The electrosurgical instrument according to claim 1, wherein the knife lockout includes an arm extending from the movable handle towards the trigger.

9. The electrosurgical instrument according to claim 8, wherein the arm defines a slot having an open end and a closed end.

10. The electrosurgical instrument according to claim 9, wherein the trigger includes a post and:
in the initial position of the movable handle, the post is received within the slot to inhibit actuation of the trigger from the un-actuated position to the actuated position,
in the grasping position of the movable handle, the post is received within the slot to inhibit actuation of the trigger from the un-actuated position to the actuated position, and
in the activated position, the post is withdrawn from the slot to permit actuation of the trigger from the un-actuated position to the actuated position.

11. A method of operating an electrosurgical instrument, comprising:
actuating a movable handle of electrosurgical instrument from an initial position to a grasping position to grasp tissue between first and second jaw members of the electrosurgical instrument;
actuating the movable handle from the grasping position to an activated position to initiate a supply energy to the first and second jaw members to treat the tissue grasped therebetween, wherein actuation the movable handle to the activated position unlocks a trigger of the electrosurgical instrument;
actuating the trigger from an un-actuated position to an actuated position to deploy a knife between the first and second jaw members to cut the grasped and treated tissue.

12. The method according to claim 11, wherein the trigger is locked in the un-actuated position prior to the movable handle reaching the activated position.

13. The method according to claim 11, wherein actuating the movable handle from the grasping position to the activated position moves the movable handle into contact with an in-line activation switch to activate the in-line activation switch thereby initiating the supply of energy to the first and second jaw members.

14. The method according to claim 11, wherein the supply of energy to the first and second jaw members is automatically terminated once it is determined that the grasped tissue is sufficiently treated.

15. The method according to claim 11, wherein the trigger is actuated from the un-actuated position to the actuated position subsequent to termination of the supply of energy.
